(19) United States
(12) Patent Application Publication
(10) Pub. No.: US 2007/0123827 A1
(43) Pub. Date: May 31, 2007

(54) Electromagnetic Field Detectable Medical Supplies

(76) Inventors: William H. Falls JR., Taipei (TW); Brian T. Mann, Houston, TX (US)

Correspondence Address: Mr. Brian T. Mann, 7D-17 TWTC, 5 Hsin Yi Road, Sec. 5, Taipei, 110, TW

(21) Appl. No.: 11/164,257

(22) Filed: Nov. 16, 2005

(51) Int. Cl. A61M 5/178

(52) U.S. Cl. 604/167.01

(57) ABSTRACT

A medical supply such as an operating room towel, laparotomy sponge, gauze pad, bandage, or swab with EMF material which eliminates the need for re-entry into a patient after operating in situations where medical supplies are missing. The patient can be easily scanned with a handheld scanner to determine if the medical supply was accidentally left inside the patient. If no EMF material is observed, unnecessary re-entry into the patient is prevented. The EMF material is incorporated into the medical supply as a tag, in the handle, as a handle, stitched to the medical supply, woven into the medical supply, or sewn in a seam in the medical supply. An identifier on, or in, the medical supply indicates that the medical supply is an EMF detectable medical supply. In addition to preventing unnecessary re-entry, scanning for an EMF detectable material is much safer than scanning for x-ray detectable material.

### BACKGROUND OF THE INVENTION

1. Field of the Invention

The present invention relates to medical supplies. More specifically, the present invention discloses Electromagnetic Field (EMF) detectable medical supplies containing a ferromagnetic material exhibiting magnetostrictive properties, such as operating towels, O.R. Towels, operating room towels, laparotomy sponges, gauze pads, scalpel, clamp, saw, scissors, needle, forceps, tweezers, and other surgical instruments, which eliminate the need for re-entry into a patient after operating, to search for missing supplies in situations where supplies are, or may be, missing.

2. Description of the Prior Art

Prior to operating, medical staff prepare the patient for surgery by placing towels on the patient around the area to be operated on. This area is commonly known as the incision area. These towels are positioned so that they overlap and surround the area where the opening will be. Additionally, the towels may be placed on trays, and surgical instruments, and absorbent medical supplies can then be laid out on the towels.

The main purpose of the towels is to establish a clean and sanitary working area during surgery, and to contain smaller amounts of spilled or splashed blood. However, during the operation or surgery, after the surgeon makes the incision, blood inevitably flows from the patient. Unfortunately, in some situations, the blood flow is substantial, and medical personnel often use the towels for other than their intended purposes.

When this occurs, there is the possibility that the towel is placed inside the body. Since the towel is drenched in blood, it may become difficult to identify as a towel. In some instances, these materials have been left inside the body of the patient. Traditionally surgical towels have not been fitted with any type of externally detectable materials, therefore, if the towels are not noticed as missing, they can remain in the patient for some time before causing enough discomfort that the patient seeks medical help. This results in the patient having to undergo another surgery to search the operation site for a missing towel, and remove the towel if found.

However, it is currently extremely difficult to determine if a towel was left inside the patient without re-entry into the patient. As a result, patients may undergo unnecessary surgery for other issues, when in reality the cause is a misplaced towel.

Furthermore, not only has the patient unnecessarily suffered, but also medical personnel are thus susceptible to malpractice lawsuits in these situations, which is extremely expensive.

Therefore, there is a need for an effective way of preventing unnecessary re-entry into a patient after surgery to determine whether towels or any other medical supplies or surgical objects were left inside the patient, thereby eliminating unnecessary patient suffering and reducing the risk of malpractice lawsuits.

In U.S. Pat. Nos. 5,456,718 and. 5,664,582, Szymaitis proposes to mark surgical instruments (surgical sponges) with a marker made exclusively of selected zero magnetostrictive, and nonmagnetostrictive soft magnetic material which will emit known specific selected high harmonic frequencies when exposed to an alternating electromagnetic field.

When studying Szymaitis' patent it becomes known that his design and use of materials is very restrictive in their use. Szymaitis uses a Beat Frequency Oscillator (BFO) type metal detector to produce the alternating electromagnetic field in his design work. Because the BFO is very inaccurate in its differentiation of materials, Szymaitis needed to use very specific and very selective nonmagnetostrictive, soft magnetic materials, so they would emit a very selective and specific oscillating frequency in order to be accurately read by the BFO detector. The use of these materials is much too restrictive. Szymaitis listed a few previously patented materials that he would prefer to use as markers, yet these materials are expensive and, in some cases, susceptible to corrosion. The use of his nonmagnetostrictive materials would also have to be remanufactured to be elongated strips encapsulated into biocompatible materials. This would greatly restrict the availability of his invention.

Also, as in McCormick's design, in U.S. Pat. No. 5,456,718, col. 6, line 60-62, Szymaitis' design could be susceptible to false readings if the patient is on a magnetic material gurney or bed. Most operating room beds and tables are made of stainless steel, aluminum or iron, all of which are magnetic materials. Also, in col. 6, line 50-53, Szymaitis disclosed that as the number of markers increases, the response of the marked object to a detector will likely be less distinctive, this would allow for false negative readings. One other major fault within Szymaitis' design is the use of elongated strips. Szymaitis failed to anticipate that once a medical supply, such as a medical sponge, is used in the body it will no longer be in a flat form but will be rolled or balled, making this elongated strip compressed and overlapping. This situation can, and will, lead to false negative indications.

Greenberg in U.S. Pat. No. 3,587,583 attempts to overcome the problems of leaving surgical objects within the body. He proposes to mark the surgical object with a permanently magnetized material. A surgeon performs an operation in the normal manner. Before closing the incision the surgeon probes for the presence of a surgical object with a magnetic field detector means which generates an electric signal which is modified in the presence of a magnetic field. If the marked object is present, the magnetic field of the magnetic marker is sensed by the magnetic field detector means which modifies the electric signal. Yet, an operating room has many types of equipment which generate permanent magnetic fields. The presence of those fields can activate the magnetic field detector means giving false detection. Because of its unreliability in an operating room, Greenberg's device is not a practical solution to the problem.

In U.S. Pat. No. 5,057,095, Fabian proposes to mark surgical implants with a marker adapted to produce identifying signal characteristics when exposed to an alternating magnetic field. He discloses three types of resonant markers that are able to resonate at a certain pre-selected frequency. The first marker is a magneto-mechanical device comprised of a permanent magnet overlaying a magnetostrictive metal strip in a plastic housing. The magnetostrictive strip vibrates when the marker is exposed to an alternating electromagnetic field and its resonance is detected when the frequency of the applied field reaches a predetermined value.

However, such devices are very sensitive to pressure and stress which will inhibit them. Since a body cavity is under some pressure and the marker may be stressed during surgery, this type of marker is not reliable for use as a marker for surgical objects.

The second proposed type is an electromechanical circuit comprised of an air coil, with or without a ferrite core and a resonant structure such as a piezoelectric crystal. As the first type, this type of marker can be adversely affected by pressure and stress because its principle of detection relies on a mechanical resonance, therefore a piezoelectric crystal type marker is also unsatisfactory.

The third type of marker proposed by Fabian is an electromagnetic LCR circuit. This type of marker can be either built out of discreet components or made of a flexible printed circuit. In the former case, this unit is expensive to build and bulky and it is impractical for surgical sponges. In the later case, due to its high electrical resonance frequency this type of marker can be adversely affected by the presence of metal objects and conductive media. Because the human body is conductive, it is also impractical for surgical sponges. Consequently, none of the markers proposed by Fabian, nor the markers of either Szymaitis or Greenberg marker, has been available on the market.

In U.S. Pat. No. 5,045,071 McCormick teaches about the use of magnetic materials for accurately locating the position of a catheter which has been inserted into a blood vessel. At column 9, lines 12-16, the patent cross references U.S. Pat. Nos. 4,41 6,289; 4,431,005 and 4,445,501 for an explanation of the general method of detection. At column 5, lines 41-52, the '005 patent explains that a distortion of the magnetic field indicates the presence of the catheter. Thus, the McCormick patent teaches that merely a change in the magnetic field is a sufficient indicator of the position of the marked object. However, McCormick's measurements can be affected by the presence of other nearby magnetic and conductive materials. Hence, McCormick's technique can and likely will provide “false positives” as to the presence or the position of the marked object.

Thus, there is a need for a marking method and apparatus for detecting surgical objects within the human body utilizing a material that can be readily identified before the patient leaves the operating suite.

### SUMMARY OF THE INVENTION

To achieve these and other advantages and in order to overcome the disadvantages of the conventional method in accordance with the purpose of the invention as embodied and broadly described herein, the present invention provides an absorbent, or solid, piece of medical supply with EMF detectable material containing a ferromagnetic material exhibiting magnetostrictive properties (heretofore known as EMF) which eliminates the need for re-entry into a patient after operating in situations where medical supplies could be or, are actually missing, or there is great patient distress following surgery and a missing object may be suspected.

Utilizing the medical supply of the present invention, the patient can easily be scanned by a specially tuned VLF metal detector, such as a handheld detector, to determine whether medical supplies have been left inside the patient after surgery. Upon scanning the body prior to closure of the incision, the electromagnetic field (EMF) detectable material can easily be observed by medical personnel if a medical supply is still inside the patient. If no EMF detectable material is observed, unnecessary re-entry into the patient is prevented.

The medical supply of the present invention comprises towels, operating room OR towels, swabs, bandages, laparotomy sponges, gauze, gauze pads, gauze bandages, medical sponges, medical bandages scalpel, clamp, saw, scissors, needle, forceps, tweezers and other surgical instruments. Each of these types of medical supplies is utilized during surgery presenting the chance or risk is high of accidentally leaving a medical supply inside of a patient.

Laparotomy sponges or Lap sponges are typically used to absorb flowing blood or blood inside the body.

Towels are used to cover Gurneys or operating beds and also provide a sterile site to lay sterilized instruments and operation tools on. The towels provide a sterile splash covering to absorb spilt or splashed blood, while also marking out an incision site. They are not typically intended to soak up blood like gauze or a lap sponge. Gauze, gauze pads, gauze bandages, medical sponges, and medical bandages are used in surgery to maintain a clean and blood free area. A scalpel, clamp, saw, scissors, needle, forceps, tweezers and other surgical instruments are used to actually perform the surgery.

The present invention provides a medical supply that comprises at least one piece of EMF detectable material so that scanning with a specially tuned VLF metal detector will indicate the presence of a medical supply.

The present invention also provides a medical supply that comprises at least one piece of EMF detectable material enclosed in a hem of the medical supply material, woven into, ironed onto, sewn into, adhered to, bonded to, or in anyway attached to the medical supply, making manufacturing convenient and ensuring the EMF detectable material is not inadvertently separated from the medical supply.

The medical supply may be, for example, an operating room towel or OR towel, a laparotomy or lap sponge, a pad, gauze, sponge, swab, or bandage. The medical supply may also be a scalpel, clamp, saw, scissors, needle, forceps, tweezers, or other surgical instrument. The material of the medical supply can be comprised of gauze, cotton, synthetic, foam, sponge, or a combination of these materials. The materials can also be metallic in nature.

The EMF detectable material (Tag) is, for example, a metallic tag made of cobalt (Co), iron (Fe), silicon (Si) and boron (B). The EMF Tag is made of a ferromagnetic material exhibiting magnetostrictive properties. Preferably, the EMF Tag is not sensitive to the orientation of other metals in the vicinity of the EMF Tag. In this way, the accuracy of reading is improved. When the EMF Tag contains the pre-selected amount of Co, Fe, Si and B it will be the only material detectable by the specially tuned VLF metal detector (detector). EMF Tags are ferromagnetic material exhibiting magnetostrictive properties that do not have their own power source and respond to the Electromagnetic Field induced into the Tag by the incoming EMF from the detector scan. The EMF from the detector is sufficient for the Tags to generate weak magnetic fields of their own. This weak EMF from the Tag is picked up and amplified by the Receiver Coil on the Detector. The magnetostrictive properties of this material are also useful in determining the specific type of medical device being monitored by the EMF detector scan.

The EMF detectable material is incorporated into the medical supply as a tag, stitched to the medical supply, woven into the medical supply, sewn into a seam of the medical supply, incorporated into a label which is then sewn into the medical supply, adhered to the handle of the medical supply, incorporated in a handle of the medical supply, or is the handle of the medical supply. Furthermore, the medical supply has, or may have an identifier that indicates the medical supply is an EMF detectable medical supply. The identifier is a label, tag, marking, printing, number, letter, shape, code, thread, or color. Typically, the identifier is visible but is not required to be. For example, the thread used in stitching a medical supply can be a different color to identify the EMF detectable medical supply. Alternatively, the color of the medical supply can be colored or marked in such a way as to identify the medical supply as an EMF detectable medical supply. The identifier may also be the magnetostrictive properties of the detectable material, by monitoring the altered characteristics of the detectable material when acted upon by an EMF producing detector.

The Detector Scanner (Detector) uses a Very Low Frequency metal detector technology known as Induction Balance. Other type of detectors can be used, however, the VLF is by far the most accurate. In a VLF detector, there are two distinct coils:
Transmitter coil—This is the outer coil loop. Within it is a coil of wire. Electricity is sent along this wire, first in one direction and then in the other, thousands of times each second. The number of times that the current's direction switches each second establishes the frequency of the unit.
Receiver coil—This inner coil loop contains another coil of wire. This wire acts as an antenna to pick up and amplify frequencies coming from target objects in the body.

The current moving through the transmitter coil creates an electromagnetic field, which is like what happens in an electric motor. The polarity of the magnetic field is perpendicular to the coil of wire. Each time the current changes direction, the polarity of the magnetic field changes. This means that if the coil of wire is parallel to the body, the magnetic field is constantly pushing down into the body and then pulling back out of it.

As the magnetic field flows back and forth into the body, it interacts with any conductive objects it encounters, causing them to generate weak magnetic fields of their own. The polarity of the object's magnetic field is directly opposite the transmitter coil's magnetic field. If the transmitter coil's field is moving downward, the object's field is moving upward.

The receiver coil is completely shielded from the magnetic field generated by the transmitter coil. However, it is not shielded from the induced magnetic fields coming from objects in the body. Therefore, when the receiver coil passes over an object giving off a magnetic field, a small electric current travels through the coil. This current is at the same frequency as the object's magnetic field. The coil amplifies the frequency and sends it to the control box of the detector, where sensors analyze the signal.

The VLF detector can distinguish between different metals. It relies on a phenomenon known as phase shifting. Phase shift is the difference in timing between the transmitter coil's frequency and the frequency of the target object. This discrepancy can result from a couple of things:
Inductance—An object that conducts electricity easily (is inductive) is slow to react to changes in the current. You can think of inductance as a deep river: Change the amount of water flowing into the river and it takes some time before you see a difference.
Resistance—An object that does not conduct electricity easily (is resistive) is quick to react to changes in the current. Using our water analogy, resistance would be a small, shallow stream: Change the amount of water flowing into the stream and you notice a drop in the water level very quickly.

Basically, this means that an object with high inductance is going to have a larger phase shift, because it takes longer to alter its induced magnetic field. An object with high resistance is going to have a smaller phase shift.

Phase shift provides VLF-based detectors with a capability called discrimination. Since most metals vary in both inductance and resistance, a VLF detector examines the amount of phase shift, using a pair of electronic circuits called phase demodulators, and compares it with the average for a particular type of metal. The detector then notifies you with an audible tone or visual indicator as to what range of metals the object is likely to be in.

VLF detectors even allow you to filter out (discriminate) objects above a certain phase shift level. Usually, you can set the level of phase shift that is filtered, generally by adjusting a knob that increases or decreases the threshold. Another discrimination feature of VLF detectors is called notching. Essentially, a notch is a discrimination filter for a particular segment of phase shift. The detector will not only alert you to objects above this segment, as normal discrimination would, but also to objects below it.

Advanced detectors even allow you to program multiple notches. For example, you could set the detector to disregard objects that have a phase shift comparable to several known metallic objects that might be found in, or around, the body, such as staples, OR Table, medical instruments, and wires. In the present invention, the detector is programmed to read only the EMF Tags of the present invention.

The present invention utilizes a Very Low Frequency (VLF) detector. The VLF detector is much more accurate in its detection and differentiation of materials and much less likely to give false positives or negative readings. Therefore, the present invention is based on the use of ferromagnetic materials with magnetostrictive properties as Tags, and not zero magnetostrictive, soft magnetic materials.

In the present invention, the Tags are mostly flat wafers, or “coins” that even when balled or rolled will still be detectable. The differences in the magnetostrictive properties are used to assist in the differentiation of the various medical supplies that could have been left inside the body cavity. Materials that are readily available and typically already FDA approved for use within the human body are used.

These and other objectives of the present invention will become obvious to those of ordinary skill in the art after reading the following detailed description of preferred embodiments.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIGS. 1A-D are drawings illustrating hemmed medical supplies with Electromagnetic Field detectable material according to embodiments of the present invention;

FIGS. 2A-D are drawings illustrating medical supplies with Electromagnetic Field detectable material according to embodiments of the present invention; and

FIGS. 3A-B are drawings illustrating medical supplies with Electromagnetic Field detectable material according to an embodiment of the present invention.

### DESCRIPTION OF THE PREFERRED EMBODIMENTS

Reference will now be made in detail to the preferred embodiments of the present invention, examples of which are illustrated in the accompanying drawings. Wherever possible, the same reference numbers are used in the drawings and the description to refer to the same or like parts.

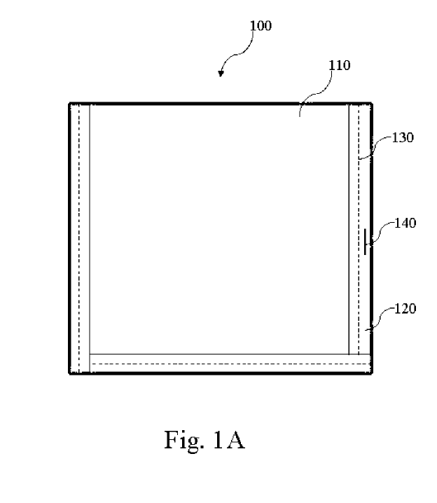

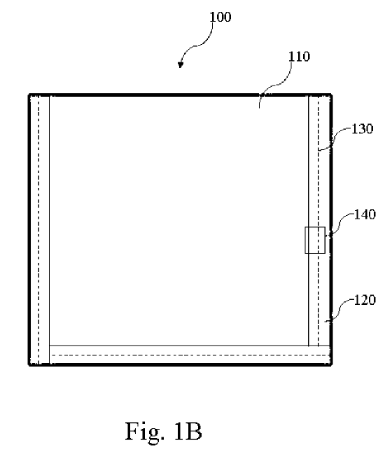

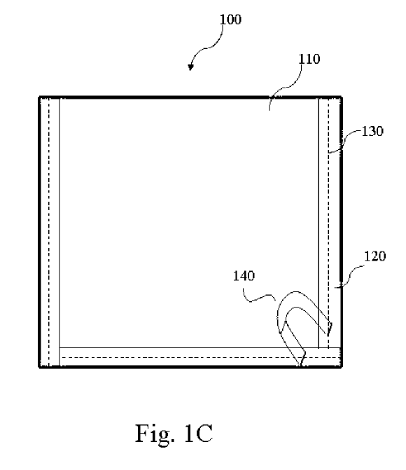

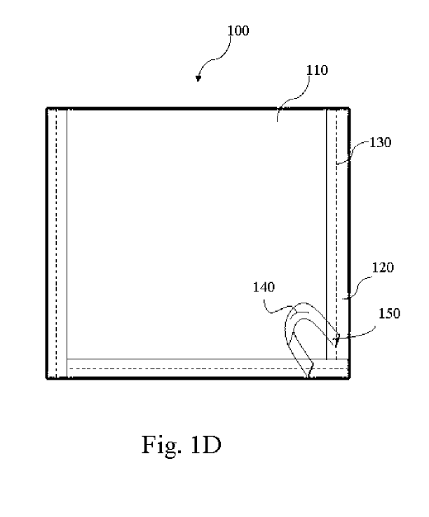

Refer to FIG. 1A, which is a drawing illustrating a hemmed medical supply with Electromagnetic Field (EMF) detectable material, containing a ferromagnetic material exhibiting magnetostrictive properties, according to an embodiment of the present invention.

As shown in FIG. 1A, the EMF detectable medical supply 100 of the present invention comprises a medical supply 110, for example, an OR towel, lap sponge, swab, bandage, or pad. The medical supply may also be a scalpel, scissors, needle, forceps, tweezers or other surgical instrument. The material of the medical supply can comprise gauze, cotton, synthetic, foam, sponge, or a combination of these materials. The material can be single-ply or multi-ply and be dyed, or un-dyed and colored as desired. The Material of the medical supply can also be metallic.

In FIG. 1A, the medical supply 110 has at least one edge of the medical supply 110 folded over and stitched to create a hem 120. In an embodiment of the present invention, three sides of the medical supply 110 are hemmed; however, one, two, three, or all four sides of the medical supply 110 can be hemmed or un-hemmed. The thread 130 used for stitching the hem 120 is for example, a synthetic thread. The color of the thread 130 can be the same color as the medical supply 110. However, to add further advantages, the color of the thread 130 can be a different color than the color of the medical supply 110. This allows the medical supply 110 to be easily identified as being EMF detectable.

Prior to stitching or during stitching, a piece of EMF detectable material 140, for example an EMF tag, is placed inside the fold in the fabric in order to be enclosed in the hem 120. The EMF detectable material 140 is a piece of material that is easily detectable upon scanning by a detector. In embodiments of the present invention the EMF detectable material 140 is an EMF tag, containing a ferromagnetic material exhibiting magnetostrictive properties. The length, size, attributes, capabilities, or amount of EMF material 140 is selectable depending on cost, effectiveness, or convenience.

Refer to FIG. 1B, which is a drawing illustrating a hemmed medical supply with Electromagnetic Field detectable material according to an embodiment of the present invention.

Alternatively, the EMF detectable material 140 is a piece that is sewn inside the hem 120 or through which the hem stitching 130 is sewn. Exposing a portion of the EMF detectable material 140 allows it to be seen without further identifying marks. Stitching through the material 140 provides further adhesion of the EMF detectable material 140 to the medical supply 150.

Refer to FIG. 1C, which is a drawing illustrating a hemmed medical supply with Electromagnetic Field detectable material according to an embodiment of the present invention.

In another embodiment the EMF detectable material 140 is attached to the medical supply 150 in the form of a loop. The thread 130 used to stitch the hem 120 attaches the EMF detectable material 140 to the medical supply 150. This has the advantage of providing a means of hanging or grasping the medical supply 150, which adds to the convenience of the medical supply.

Refer to FIG. 1D, which is a drawing illustrating a hemmed medical supply with Electromagnetic Field detectable material according to an embodiment of the present invention.

In another embodiment of the present invention, the EMF detectable material 140 is added to a loop 150 attached to the medical supply 150. The EMF detectable material 140 can be enclosed in, sewn to, attached to, or adhered to the loop 150. This allows the EMF detectable material 140 to be attached to the medical supply 150 at any time.

The EMF detectable material can be shaped, coded, numbered, or have identifying characteristics. In situations where other EMF detectable objects are intentionally placed inside a patient, the unique phase shift characteristics of the EMF detectable material easily identifies the medical supply of the present invention. For example, the material can have an exclusive EMF phase shift that can be read upon scanning.

Additionally, the material can be a label or inside a label attached to the medical supply. For example, a label with the manufacturer or brand name of the medical supply can comprise the EMF detectable material. This label indicates the medical supply maker and identifies the medical supply as being EMF detectable.

Some medical supplies are disposable but others are re-usable. The medical supplies can be sterilized, for example by Gamma radiation, autoclave, or steam, or used un-sterilized. Furthermore, the medical supplies can be colorized or color-coded to indicate whether they are disposable, re-usable, sterilized, or un-sterilized. The medical supplies can also be colorized or color-coded to indicate how and where they're to be used in a hospital or other medical clinic.

Rather than enclosing the EMF detectable material in the hem, the material can be sewn, folded into, woven into or otherwise adhered to the medical supply.

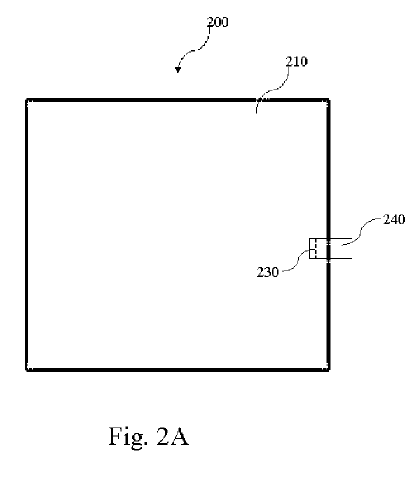

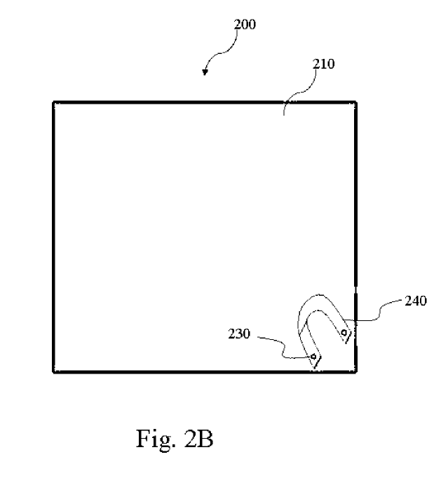

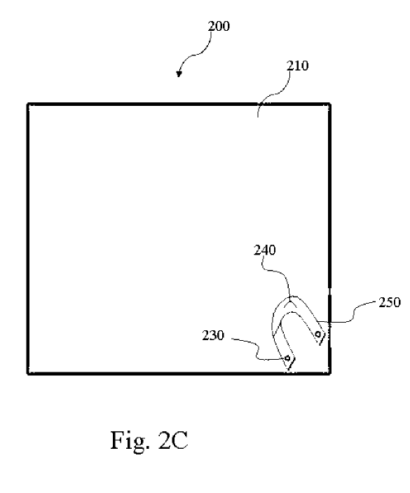

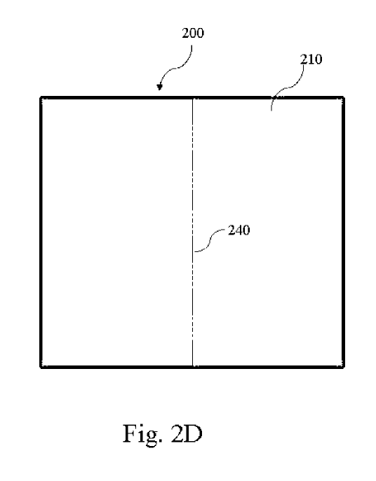

Refer to FIG. 2A, which is a drawing illustrating a medical supply with Electromagnetic Field detectable material according to an embodiment of the present invention.

Certain medical supplies are not hemmed as those illustrated in FIGS. 1A-D. As shown in FIG. 2A, the EMF detectable medical supply 200 of the present invention comprises an un-hemmed medical supply 210 and an EMF detectable material 240 connected to the medical supply 210 by an attaching means 230. For example, the EMF detectable material 240 can be stitched by thread to the medical supply 230. However, any other connecting means such as adhesive, bonding, folding, or weaving can be used. Additionally, the EMF detectable material 240 can comprise a label or be enclosed in a label or tag. This also allows the medical supply to be easily grasped by hospital personnel or simply be easily identified as having the EMF detectable material in on it.

Refer to FIG. 2B, which is a drawing illustrating a medical supply with Electromagnetic Field detectable material according to an embodiment of the present invention.

In this embodiment of the present invention the EMF detectable material 240 is attached to the medical supply 210 by the connecting means 230 in the shape of a loop. This allows the medical supply 210 to be easily grasped or hung.

Refer to FIG. 2C, which is a drawing illustrating a medical supply with Electromagnetic Field detectable material according to an embodiment of the present invention.

As shown in FIG. 2C, the EMF detectable material 240 is attached to, encased in, folded in, woven in, or adhered to, a loop 250 attached to the medical supply 210. The loop 250 is attached to the medical supply 210 by connecting means 230.

Refer to FIG. 2D, which is a drawing illustrating a medical supply with Electromagnetic Field detectable material according to an embodiment of the present invention.

As shown in FIG. 2D, the EMF detectable material 240 may also be woven, or folded, into the medical supply 210 as a line going through it, or in a manner that is readily identifiable by scanning. For example, the EMF detectable material can be similar to a thread and stitched, folded or woven into the medical supply.

Rather than enclosing the EMF detectable material in the hem, the material can be sewn, folded into, woven into or otherwise adhered to the medical supply.

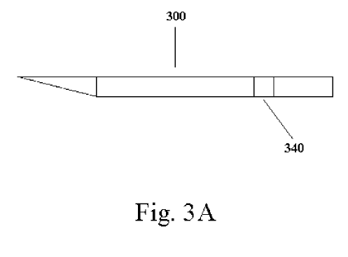

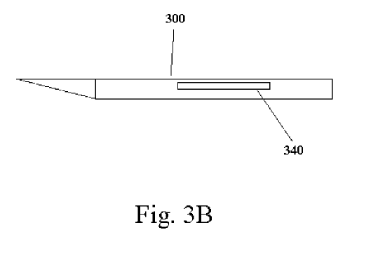

Refer to FIG. 3A, which is a drawing illustrating a medical supply with Electromagnetic Field detectable material according to an embodiment of the present invention.

As shown in FIG. 3A, the EMF detectable material 340 is attached to, or adhered to the medical supply 300 in a manner that is readily identifiable by scanning.

Refer to FIG. 3B, which is a drawing illustrating a medical supply with Electromagnetic Field detectable material according to an embodiment of the present invention. As shown in FIG. 3B, the EMF detectable material 340 is attached to, encased in, or adhered to the medical supply 300 in a manner that is readily identifiable by scanning.

After surgery or prior to closure of the incision in the patient, medical personnel can quickly scan the patient using the detector to ensure that all medical supplies have been removed from the body. In this way, no medical supplies will be inadvertently left inside the patient.

As described above, the present invention provides a medical supply with EMF detectable material, which eliminates the need for re-entry into a patient after operating in situations where medical supplies are missing.

Utilizing the Medical Supplies of the present invention, the patient can easily be scanned to determine whether a medical supply has been left inside the patient after surgery. Upon scanning, the EMF detectable material can easily be observed by medical personnel if a Medical Supply is still inside the patient. If no EMF detectable material is observed, unnecessary re-entry into the patient is prevented thereby reducing a patient's suffering and the risk of malpractice lawsuits. Additionally, utilizing EMF detectable material instead of other detectable materials prevents unnecessary exposure to chemicals or radiation such as x-ray radiation. This further enhances the safety of the EMF detectable medical supply of the present invention.

It will be apparent to those skilled in the art that various modifications and variations can be made to the present invention without departing from the scope or spirit of the invention. In view of the foregoing, it is intended that the present invention cover modifications and variations of this invention provided they fall within the scope of the invention and its equivalent.

What is claimed is:

1. An Electromagnetic Field (EMF) detectable medical supply comprising;
a piece of medical supply; and
at least one piece of Electromagnetic Field (EMF) detectable material containing a ferromagnetic material exhibiting magnetostrictive properties connected to the medical supply, the Electromagnetic Field detectable material allowing indication of the medical supply upon scanning by an EMF detector.

2. The Electromagnetic Field detectable medical supply of claim 1, the medical supply comprising an operating room surgical towel, a laparotomy sponge, a pad, gauze, cotton, swab, bandage, scalpel, scissors, clamp, saw, needle, forceps, tweezers or other surgical instrument.

3. The Electromagnetic Field detectable medical supply of claim 1, the Electromagnetic Field detectable material comprising an Electromagnetic Field tag comprising Cobalt, Iron, Silicon, or Boron or a combination of these, the Tag comprising a ferromagnetic material exhibiting magnetostrictive properties.

4. The Electromagnetic Field detectable medical supply of claim 3, the Electromagnetic Field tag comprising a flat film form, wafer, coin, strip, a wire incorporated into a pliable PVC jacket, or impregnated into a PVC or cotton thread, the Electromagnetic Field tag producing an electromagnetic field and connected to the medical supply for the purpose of producing an EMF when scanned by an EMF detector.

5. The Electromagnetic Field detectable medical supply of claim 1, wherein the Electromagnetic Field detectable material is incorporated into a tag or label, stitched to the medical supply, sewn into a seam of the medical supply, folded directly into the medical supply, woven directly into the medical supply, incorporated in a handle of the medical supply, adhered to the medical supply, is the handle of the medical supply, or is incorporated into the structure of the medical supply.

6. The Electromagnetic Field detectable medical supply of claim 5, whereby the stitching uses a thread of a different color than the color of the medical supply.

7. The Electromagnetic Field detectable medical supply of claim 1, the medical supply comprising cotton, synthetic material, gauze, foam, sponge, a metallic material, or a combination of these.

8. The Electromagnetic Field detectable medical supply of claim 1, the Electromagnetic Field detectable material comprising shaped, coded, magnetostrictive coded, data, numbered, or identifying characteristics to identify a scanned object as a medical supply.

9. The Electromagnetic Field detectable medical supply of claim 1 further comprising an identifier on, or in, the medical supply that indicates the medical supply is a Electromagnetic Field detectable medical supply.

10. The Electromagnetic Field detectable medical supply of claim 9, the identifier comprising a label, thread, tag, marking, printing, number, letter, shape, code, magnetostrictive code, or color.

11. The Electromagnetic Field detectable medical supply of claim 9, wherein the identifier is visible.

12. An Electromagnetic Field detectable medical supply comprising;
a piece of medical supply;
at least one piece of Electromagnetic Field detectable material connected to the medical supply, the Electromagnetic Field detectable material comprising a ferromagnetic material exhibiting magnetostrictive properties, allowing indication of the medical supply upon scanning; and
an identifier on, or in, the medical supply that indicates the medical supply is an Electromagnetic Field detectable medical supply.

13. The Electromagnetic Field detectable medical supply of claim 12, the medical supply comprising an operating room towel, a laparotomy sponge, a pad, gauze, cotton, swab, bandage, scalpel, clamp, saw, scissors, needle, forceps, tweezers or other surgical instrument.

14. The Electromagnetic Field detectable medical supply of claim 12, the Electromagnetic Field detectable material comprising ferromagnetic material exhibiting magnetostrictive properties in the form of a metallic tag, strip or ribbon or other ferromagnetic material exhibiting magnetostrictive properties that would produce an EMF signal.

15. The Electromagnetic Field detectable medical supply of claim 12, wherein the Electromagnetic Field detectable material is connected to the medical supply by incorporating into a tag or label, and stitching to the medical supply.

16. The Electromagnetic Field detectable medical supply of claim 12, wherein the Electromagnetic Field detectable material is stitched onto or into the medical supply, incorporated into a tag or label, sewn into a seam of the medical supply, woven into the medical supply, incorporated in a handle of the medical supply, adhered to the medical supply, or is in, on or part of the medical supply.

17. The Electromagnetic Field detectable medical supply of claim 15, whereby the stitching uses a thread of a different color than a color of the medical supply.

18. The Electromagnetic Field detectable medical supply of claim 12, the medical supply comprising cotton, synthetic material, gauze, foam, sponge, a metallic material, or a combination of these.

19. The Electromagnetic Field detectable medical supply of claim 12, the Electromagnetic Field detectable material comprising shaped, coded, magnetostrictive coded, data, numbered, or identifying characteristics to identify a scanned object as a medical supply.

20. The Electromagnetic Field detectable medical supply of claim 12, the identifier comprising a label, thread, tag, marking, printing, number, letter, shape, code, magnetostrictive code, or color.

21. The Electromagnetic Field detectable medical supply of claim 12, wherein the identifier is visible.

22. The Electromagnetic Field detectable medical supply of claim 12, wherein the identifier is incorporated into the medical supply and is not visible.

23. An Electromagnetic Field detectable medical supply comprising;
a piece of medical supply;
at least one piece of Electromagnetic Field detectable material connected to the medical supply, the Electromagnetic Field detectable material comprising a ferromagnetic material exhibiting magnetostrictive properties, allowing indication of the medical supply upon scanning by a Very Low Frequency detector programmed to read only the Electromagnetic Field detectable material, the Electromagnetic Field detectable material comprising Cobalt, Iron, Silicon, Boron, or a combination of these; and
a visible identifier on, or in, the medical supply that indicates the medical supply is an Electromagnetic Field detectable medical supply.
